(19) United States
(12) Plant Patent Application Publication
(10) Pub. No.: US 2007/0074320 P1
(43) Pub. Date: Mar. 29, 2007

(54) Forsythia plant named 'Courtacour'

(75) Inventor: M. Alain Cadic, Beaucouze (FR)

Correspondence Address: BUCHANAN, INGERSOLL & ROONEY PC, POST OFFICE BOX 1404, ALEXANDRIA, VA, 22313-1404, US

(73) Assignee: AGRI OBTENTIONS S.A., Guvancourt cedex (FR)

(21) Appl. No.: 11/606,235

(22) Filed: Nov. 30, 2006

Related U.S. Application Data
(63) Continuation of application No. 11/071,159, filed on Mar. 4, 2005.
Application No. 11/071,159 is a continuation of application No. 10/288,510, filed on Nov. 6, 2002, now abandoned.
Application No. 10/288,510 is a continuation of application No. 09/607,278, filed on Jun. 30, 2000, now abandoned.

(51) Int. Cl. A01H 5/00

(52) U.S. Cl. PLT/230

(57) ABSTRACT

A new and distinct cultivar of Forsythia plant is provided that is a seedling formed following the induced mutation of the ‘Spring Glory’ cultivar (non-patented in the United States). Gamma radiation was utilized to create the mutation. The new cultivar can be readily distinguished from the parent cultivar by the presence of a very compact dwarf growth habit with exceptionally short internodes. Attractive bright yellow flowers are formed on an abundant basis. In view of the short internodes the blossoms well cover the relatively thick branches. Dark green foliage begins to appear during flowering. The winter hardiness is good. The plant is particularly well suited for growing in containers. The new cultivar also is well suited for growing as attractive ornamentation in the landscape.

This is a Continuation of application Ser. No. 11/071,159, filed Mar. 4, 2005 which is a Continuation of application Ser. No. 10/288,510, filed Nov. 6, 2002 (now abandoned), which is a Continuation of application Ser. No. 09/607,278, filed Jun. 30, 2000 (now abandoned).

### BOTANICAL/COMMERCIAL CLASSIFICATION

Forsythia×intermedia/Forsythia Plant

### VARIETAL DENOMINATION

cv. ‘Courtacour’

### SUMMARY OF THE INVENTION

A new and distinct variety of Forsythia×intermedia plant is provided that is a mutation derived from the ‘Spring Glory’ cultivar (non-patented in the United States).

When creating the new cultivar of the present invention plants of the ‘Spring Glory’ cultivar were irradiated with gamma rays, seeds were formed thereon following open pollination, and the seedlings produced upon the planting of such seeds were observed. The irradiation and the study of the resulting seedlings were carried out at the Institut National De La Recherche Agronomique located at Angers, France.

It was found that a single plant observed following such irradiation and the planting of seeds possessed the characteristics of the Forsythia cultivar of the present invention. The characteristics of such new cultivar can be summarized as follows:
(a) exhibits a very compact dwarf upright spreading growth habit with exceptionally short internodes,
(b) forms attractive bright yellow flowers on an abundant basis,
(c) forms dark green foliage that begins to appear during flowering,
(d) exhibits good winter hardiness, and
(e) is well suited for growing in containers.

The new cultivar of the present invention well meets the needs of the horticultural industry and is particularly well suited for growing as attractive ornamentation. The bright yellow blossoms are formed in abundance in the springtime and in view of the short internodes well cover the relatively thick branches. Adjoining blossoms commonly touch and overlap each other. Even young plants are very floriferous.

The size of the plant at maturity is considerably smaller than that of the ‘Spring Glory’ cultivar. The new cultivar grows well in containers, tubs, and pots and can be grown on balconies and terraces. Alternatively, it can be grown to advantage in the landscape where an attractive smaller-sized ornamental plant is desired.

Vegetatively propagated plants of the new cultivar commonly first flower at an age of approximately one year. The volume of such flowering commonly increases as the plants reach maturity at an age of approximately three to four years.

The new cultivar has been found to readily undergo asexual propagation by the use of softwood cuttings to produce plants that are true to type. Roots readily form on such cuttings during the summer. Such asexual propagation has been carried out at Angers, France, and elsewhere.

The new cultivar has been named the ‘Courtacour’ cultivar.

### BRIEF DESCRIPTION OF THE PHOTOGRAPHS

The new cultivar that is depicted was approximately ten years of age and was being grown outdoors at Angers, France. Color is shown as true as is reasonably possible in photographs of this character.

FIG. 1 shows a typical plant when blossoming during the springtime. The dwarf upright spreading growth habit and profusion of bright yellow flowers closely borne along branches are apparent.

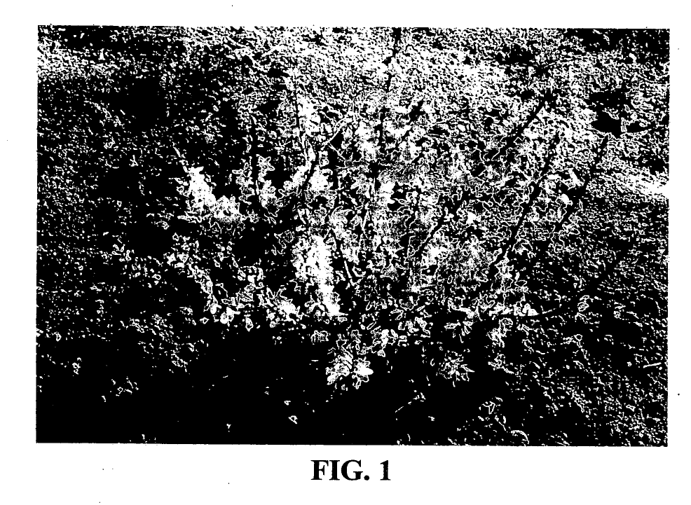

FIG. 2 shows fruit and seeds on Nov. 17, 2001 in a dehydrated state following drying at room temperature.

### DETAILED DESCRIPTION

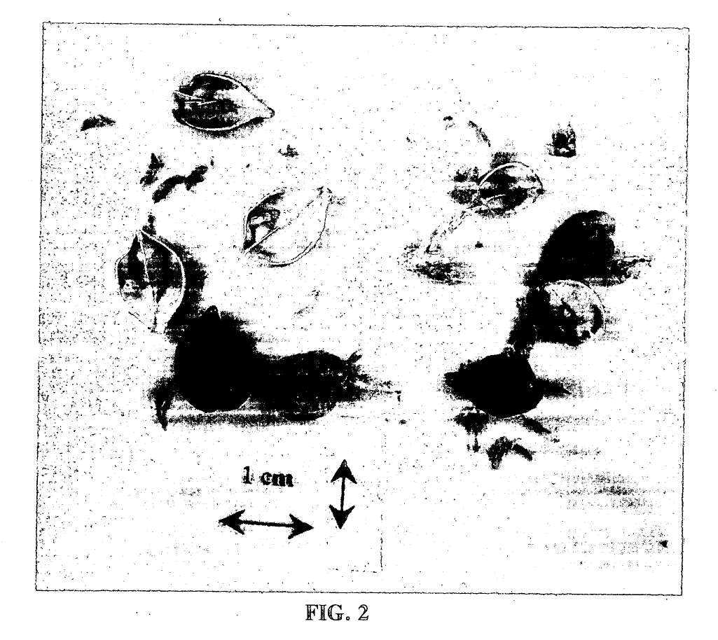

The following description is based on the observation of plants of the new cultivar growing outdoors at Angers, France. Such plants had been asexually reproduced through the use of softwood cuttings and were approximately ten years of age. The color terminology utilized in the description that follows is to be accorded its ordinary dictionary significance. Reference to the R.H.S. Colour Chart of The Royal Horticultural Society, London, England, sometimes is included.
Origin: Seedling produced following the induced mutation of the ‘Spring Glory’ cultivar (non-patented in the United States). The ‘Spring Glory’ cultivar was irradiated with gamma rays from cobalt 60.
Parentage: Seedling of ‘Spring Glory’.
Classification: Forsythia×intermedia, cv. ‘Courtacour’.
Plant:
   Form.—well-branched flowering shrub.
   Habit.—erect, upright, spreading, and very compact. The plant is considered to be a dwarf unlike its ‘Spring Glory’ parent.
   Internode length.—exceptionally short, considerably shorter than that of the parent ‘Spring Glory’ cultivar. Typical internode lengths are approximately 20 to 40 mm.
   Height.—approximately 0.5 to 0.8 m at maturity, and considerably shorter than the ‘Spring Glory’ cultivar. The ‘Spring Glory’ cultivar commonly will assume a height of approximately 1.5 m.
   Width.—approximately 1 m at maturity.
Foliage:
   Disposition.—opposite, and commonly begins to appear during flowering.
   Configuration.—ovate and simple.
   Size—commonly approximately 5.4 cm in length and approximately 2.2 cm in width.
   Color.—near Yellow-Green Group 147A on the upper surface and near Yellow-Green Group 147B on the under surface.
   Petiole—commonly approximately 1.5 cm in length.
Inflorescence:
   Configuration.—more compact than those of the ‘Spring Glory’ cultivar and bell-shaped.
   Size.—approximately 18.4 mm.
   Color.—bright yellow, Yellow Group 12A. This compares to Yellow Group 7A for the parent ‘Spring Glory’ cultivar.
   Sepals.—four in number, approximately 4.5 mm in length, and near Yellow-Green Group 144A on the upper surface and near Yellow-Green Group 144B on the under surface. The total length of the calyx commonly is approximately 6 mm.
   Petals.—elliptic in configuration, four in number, and shorter than those of ‘Spring Glory’. Commonly approximately 14.6 mm in length and approximately 6.7 mm in width.
   Disposition.—closely spaced along the branch (as illustrated) in view of exceptionally short internode length, and are borne on short pedicels.
   Peduncle.—commonly approximately 5.5 mm in length.
   Pedicel.—near Yellow-Green Group 144B in coloration.
   Stamen.—commonly approximately 5.4 mm in length, and pale greenish-yellow to near white in coloration. Such coloration is extremely difficult to further characterize through the use of a color chart.
   Filaments.—commonly approximately 3.7 mm in length, and near Yellow Group 2D in coloration.
   Anthers.—two in number, tend to extend above the stigma, and near Yellow Group 4D in coloration.
   Pollen.—present, near Yellow Group 4D in coloration, and commonly approximately 28.8 microns in size when dried.
   Pistil.—commonly approximately 3.4 mm in length.
   Time of flowering.—springtime, and approximately 15 days later than the ‘Spring Glory’ cultivar. The time of flowering is influenced by the intensity of the winter frost and the springtime temperatures that are experienced. In 2000 blossoming occurred at the end of March. The duration of flowering commonly is approximately 2 to 3 weeks depending on the climatic conditions.
   Petal drop.—medium tendency to detach.
   Fruit.—the plant is fertile and forms ovoid fruits in a sparse quantity having a plurality of internal compartments. Such fruit commonly displays a rough surface and is approximately 1.1 cm in length on average and approximately 0.8 cm in width on average. The upper surface is near Greyed-Orange Group 165A and the under surface is near Greyed-Orange Group 166C. See FIG. 2.
   Seeds.—commonly approximately four bean-shaped seeds (as illustrated) per fruit having a length of approximately 4 mm and a width of approximately 1 mm, and a coloration when dried of near Greyed-Orange Group 166C. See FIG. 2.
Growing conditions: Does well in full sun or semi-shade, and prefers soil that is not exceedingly dry.
Hardiness: Very good. Has withstood temperatures of −25° C. to −30° C.
Propagation: Can be readily propagated while utilizing softwood cuttings.
Usage: Attractive erect ornamental spring-flowering shrub for growing in pots, tubs or containers in small gardens, balconies and terraces. Alternatively can be grown in the landscape. Requires little care.

1. A new and distinct Forsythia that is derived from the ‘Spring Glory’ (non-patented in the United States) cultivar having the following combination of characteristics:
(a) exhibits a very compact dwarf upright spreading growth habit with exceptionally short internodes,
(b) forms attractive bright yellow flowers on an abundant basis,
(c) forms dark green foliage that begins to appear during flowering,
(d) exhibits good winter hardiness, and
(e) is well suited for growing in containers;
substantially as shown and described.
